(19) United States
(12) Patent Application Publication
(10) Pub. No.: US 2005/0131388 A1
(43) Pub. Date: Jun. 16, 2005

(54) Embedded spinal injector

(76) Inventor: Henrich Cheng, Taipei (TW)

Correspondence Address: Jackson Walker L.L.P.;Suite 2100, 112 E. Pecan Street, San Antonio, TX, 78205, US

(21) Appl. No.: 10/737,479

(22) Filed: Dec. 16, 2003

(51) Int. Cl. A61K 9/22

(52) U.S. Cl. 604/891.1

(57) ABSTRACT

An embedded spinal injector has a reservoir compartment, a pressing compartment in communication with the reservoir compartment, a testing compartment connected to the pressing compartment and having a unidirectional valve sandwiched between the pressing compartment and the testing compartment to stop reverse flow of medicine from the pressing compartment. A hole is defined in the first inner casing and an opening is defined in the second inner casing such that refill of the reservoir can be completed by applying a needle extending through the outer casing and a portion of the first inner casing corresponding to hole. Deformation of a portion of the outer casing corresponding to the opening of the second inner casing is able to force medicine received in the pressing compartment to flow to the testing compartment and the outlet in communication with the testing compartment.

### BACKGROUND OF THE INVENTION

1. Field of the Invention

The present invention relates to an injector, and more particularly to an embedded spinal injector to release medicine received in the injector to the spine so as to lessen pain of the patient.

2. Description of Related Art

Patients in great pain often need morphine to relieve the pain. Doctors use hypodermic needles to inject pain killer to release the pain. However, when the pain comes in a periodical cycle or it can be foreseen that the patient will suffer from such pain for a long period of time, doctors will adopt an embedded injector. The embedded injector (4), as shown in FIG. 4, is received in the patient's spine and has a dome shaped outer casing (41) which is made of silicone and an inner casing (42) mounted inside the outer casing (41) and having an opening (43). An outlet (44) in communication with an inner space of the inner casing (42) is provided on a bottom of the embedded injector (4) and extended outwardly from the outer casing (41). It is known that silicone is soft in nature and preferably the inner casing (42) is made of plastic. Therefore, after the embedded injector (4) is received in the patient's spine, the patient is able to press, via the skin covering the injector (4), a portion of the outer casing (41) corresponding to the opening (43) of the inner casing (42) such that the pain killer (not shown) in the inner casing (42) is forced to be injected out of the injector to ease the patient's pain. From the structure of the conventional embedded injector (4), it is learned that everytime the patient presses the outer casing (41) to force the pain killer inside the inner casing (42) out of the embedded injector (4), the doctor will have to use a needle to refill the inner casing (42) with fresh pain killer, which is quite troublesome and labor inefficient.

To overcome the shortcomings, the present invention tends to provide an improved embedded spinal injector to mitigate the aforementioned problems.

### SUMMARY OF THE INVENTION

The primary objective of the present invention is to provide an improved embedded spinal injector such that medicine can be stored in the reservoir compartment and the pressing compartment. Once the medicine in the pressing compartment is forced out of the spinal injector of the present invention, fresh medicine will be automatically supplied to the pressing compartment from the reservoir compartment. Thus, multiple doses of painkiller can be accessed from a single injection of the painkiller into the reservoir compartment, whereby pain and injury from repeated injections are avoided.

Another objective of the present invention is to provide a testing compartment in communication with the pressing compartment such that paramedical personnel is able to withdraw body fluid received in the testing compartment for further test without the need of additional operation to have access to the body fluid.

Other objects, advantages and novel features of the invention will become more apparent from the following detailed description when taken in conjunction with the accompanying drawings.

### BRIEF DESCRIPTION OF THE DRAWINGS

FIG. 1 is a cross sectional side plan view showing the embedded spinal injector of the present invention;

FIG. 2 is a schematic side view showing that a needle is applied to refill the reservoir compartment with medicine;

FIG. 3 is a schematic side view showing that the pressing compartment is pressed to force the medicine received in the pressing compartment to flow out of the spinal embedded injector from the outlet; and

FIG. 4 is a cross sectional view of a conventional spinal injector.

### DETAILED DESCRIPTION OF THE PREFERRED EMBODIMENT

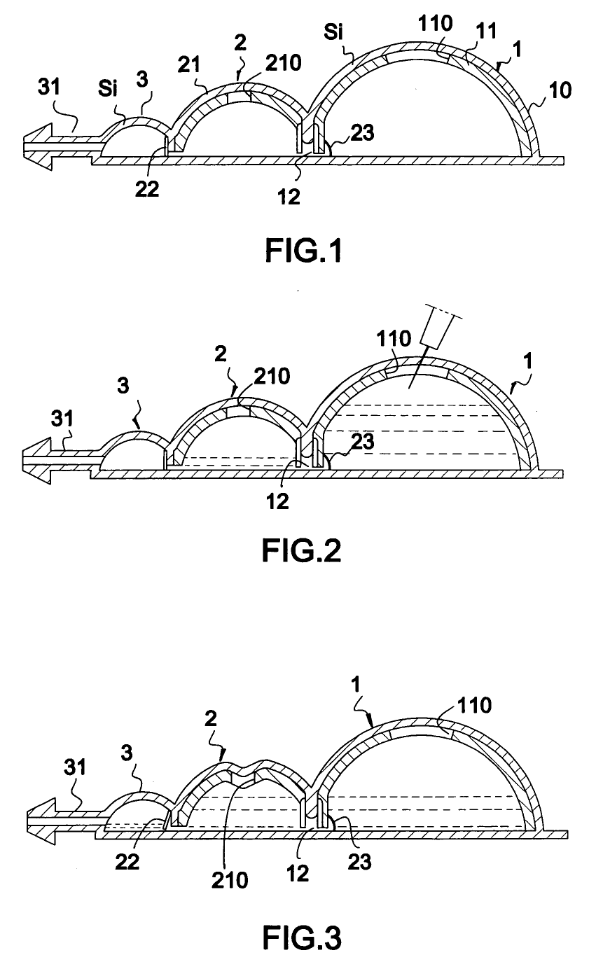

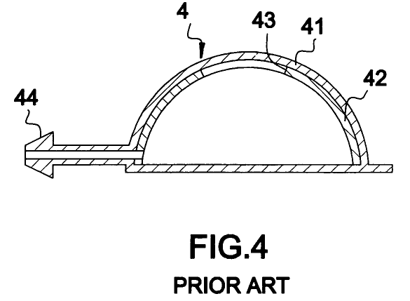

With reference to FIG. 1, an embedded spinal injector in accordance with the present invention has a reservoir compartment (1), a pressing compartment (2) and a testing compartment (3) with an outlet (31) extending out of the testing compartment (3).

The embedded spinal injector of the present invention has an outer casing (10) made of silicone and covering both the reservoir compartment (1) and the pressing compartment (2). The reservoir compartment (1) has a dome-shaped first inner casing (11) securely attached to an inner face of the outer casing (10) and having a hole (110).

The pressing compartment (2) has a dome-shaped second inner casing (21) securely attached to a portion of the inner face of the outer casing (10) and having an opening (210).

The testing compartment (3) is formed by a portion of the outer casing (10) such that the testing compartment (3) is soft in nature. The outlet (31) is securely connected to and in communication with the testing compartment (3).

It is noted that a passage (12) is defined between the reservoir compartment (1) and the pressing compartment (2) so that the reservoir compartment (1) communicates with the pressing compartment (2). Further a unidirectional valve (22) is formed between the pressing compartment (2) and the testing compartment (3) to prevent reverse flow of medicine from the testing compartment (3) to the pressing compartment (2). A second unidirectional valve (23) is provided between the reservoir compartment (1) and the pressing compartment (2) and attached to the inner side face of the reservoir compartment (1) such that when the reservoir compartment (1) is filled with medicine, the medicine is able to flow to the pressing compartment (2) and reverse flow from the pressing compartment (2) to the reservoir compartment (1) is prevented.

With reference to FIG. 2, when the embedded spinal injector of the present invention is in application, a needle is applied to inject medicine into the reservoir compartment (1) by extending through the outer casing (10) and the hole (110) of the first inner casing (11). Due to the communication between the reservoir compartment (1) and the pressing compartment (2), the medicine will flow into and fill the pressing compartment (2). Because of the unidirectional valve (22) between the pressing compartment (2) and the pressing compartment (3), the medicine inside the pressing compartment (2) will not flow to the testing compartment (3).

With reference to FIG. 3, when the patient needs the medicine inside the spinal injector of the present invention, the patient presses a portion of the outer casing (10) corresponding to the opening (210) to allow the medicine inside the pressing compartment (2) to force the unidirectional valve (22) open. Thus the medicine is able to flow out of the spinal injector from the outlet (31). It is appreciated that due to the provision of the second unidirectional valve (23) between the reservoir compartment (1) and the pressing compartment (2), the medicine flowing to the testing compartment (3) will not flow to the reservoir compartment (1). It is noted that the reservoir compartment (1) has a dimension larger than that of the pressing compartment (2) such that when the medicine inside the pressing compartment (2) is forced out, the medicine inside the reservoir compartment (1) will automatically flow into the pressing compartment (2) to refill the pressing compartment (2) in order to achieve pressure balance. Meanwhile, after the pressure from the outflow medicine is released, the unidirectional valve (22) closes the communication between the pressing compartment (2) and the testing compartment (3) and thus the medicine flowing from the reservoir compartment (1) is able to be stored in the pressing compartment (2) for subsequent use.

While the medicine is flowing out of the spinal injector of the present invention, some body fluid may flow into the testing compartment (3) and is thus received inside the testing compartment (3). Therefore, the paramedical personnel may withdraw the body fluid in the testing compartment (3) for further examination purpose.

Because the reservoir compartment (1) has a receiving space larger that that of the pressing compartment (2), the paramedical personnel may save time and effort by not having to repeatedly inject medicine for relieving the pain.

It is concluded that the structure of the present invention is simple and thus the manufacture cost is low. Furthermore, due to the provision of the reservoir compartment (1) and the testing compartment (3), the patient has ample medicine inside the reservoir compartment (1) to allow multiple dosages of painkiller to be given without the patient suffering the pain from the needle for medicine refill.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

1. An embedded spinal injector comprising:
an outer casing;
a reservoir compartment having a first inner casing securely attached to a portion of an inner face of the outer casing and a hole defined in the first inner casing; and
a pressing compartment adjacent to the reservoir compartment and having a second inner casing securely attached to a portion of the inner face of the outer casing and, an opening defined in the second inner casing and an outlet provided to communicate with the pressing compartment,
wherein the reservoir compartment has a dimension larger that that of the pressing compartment and a deformation of a portion of the outer casing corresponding to the opening is able to force the medicine to flow to the outlet and patient's spine.

2. The embedded spinal injector as claimed in claim 1, wherein the reservoir compartment and the pressing compartment are dome-shaped.

3. The embedded spinal injector as claimed in claim 2, wherein a passage is defined between the reservoir compartment and the pressing compartment so as to communicate the reservoir compartment with the pressing compartment.

4. The embedded spinal injector as claimed in claim 2 further comprising a testing compartment sandwiched between the pressing compartment and the outlet for receiving a patient's body fluid.

5. The embedded spinal injector as claimed in claim 4, wherein a unidirectional valve is provided between the pressing compartment and the testing compartment to stop reverse flow of medicine from the pressing compartment.

6. The embedded spinal injector as claimed in claim 5, wherein a second unidirectional valve is provided between the reservoir compartment and the pressing compartment to stop reverse flow of medicine from the reservoir compartment.
